(19) United States
(12) Patent Application Publication
(10) Pub. No.: US 2020/0163931 A1
(43) Pub. Date: May 28, 2020

(54) ORAL COMPOSITION OF EXTRACTED CANNABINOIDS AND METHODS OF USE THEREOF

(71) Applicant: Columbia Care LLC, New York, NY (US)

(72) Inventor: Aaron Dely, Miami, FL (US)

(21) Appl. No.: 16/340,436

(22) PCT Filed: Oct. 11, 2017
(86) PCT No.: PCT/US2017/056201

§ 371 Date: Apr. 9, 2019

Related U.S. Application Data
(60) Provisional application No. 62/406,980, filed on Oct. 12, 2016.

(51) Int. Cl. A61K 31/352; A61K 36/185; A61K 9/16; A61K 9/48; A61K 31/05; A61K 9/00

(52) U.S. Cl. CPC A61K 31/352; A61K 36/185; A61K 9/1652; A61K 9/0053; A61K 31/05; A61K 9/1623; A61K 9/48

(57) ABSTRACT

The present invention provides oral pharmaceutical compositions comprising the release of at least two cannabinoids in the treatment of disorders or the symptoms thereof. A method of manufacture for their preparation and methods of use thereof are also provided.

### CROSS REFERENCE TO RELATED APPLICATION

This application is a §371 national-stage application based on Patent Cooperation Treaty Application serial number PCT/US2017/056201, filed Oct. 11, 2017; which claims the benefit of priority to U.S. Provisional Application No. 62/406,980, filed on Oct. 12, 2016, the disclosure of which is herein incorporated by reference.

### BACKGROUND OF THE INVENTION

### 1. Field of the Invention

The present disclosure relates to oral pharmaceutical compositions comprising extracted cannabinoids in combination for medical treatments.

### 2. Description of the Prior Art

The medicinal and psychoactive properties of the cannabis plant have been known for centuries. While it has been illegal in many countries, there is a growing populous to lobby for legalization of its use, especially for medicinal purposes.

Cannabis is believed to provide benefits in the treatment of multiple disorders with safer and fewer serious side effects than most prescription drugs currently used as anti-emetics, muscle relaxants, hypnotics and analgesics, and the like. A disadvantage in treating patients with cannabis is the psychoactive effect, especially in “naive” cannabis users. Furthermore, there have been reports of unpleasant reactions to cannabis, such as anxiety, panic or hallucinations. It is believed that the undesirable side effects are most commonly associated with higher doses of cannabis, and are related to the difficulty in controlling the dosage when the drug is smoked or eaten in cannabis-enriched confectionaries.

Cannabis has also been used to treat the symptoms in patients suffering from serious medical conditions. For example, cannabis has been used to alleviate symptoms associated with cancer, anorexia, AIDS, chronic pain, muscle spasticity, glaucoma, arthritis, migraine and many other illnesses. Cannabis is recognized as having anti-emetic properties and has been successfully used to treat nausea and vomiting in cancer patients undergoing chemotherapy. Cannabis has also been reported in treating the weight loss syndrome of AIDS and for the treatment of glaucoma by reducing intraocular pressure. Cannabis is also known for its muscle relaxing and anti-convulsant effects.

The most prevalent mode of administration of medical cannabis is by smoking. This mode of administration can have adverse effects on the lungs. Cannabis smoke carries more tar and other particulate matter than tobacco, and may be a cause of lung diseases including lung cancer. Furthermore, many patients find the act of smoking unappealing, as well as generally unhealthy.

Accordingly, there is significant interest in developing other means to administer cannabis to patients.

U.S. Patent Application No. 2012/0231083 discloses an oral formulation of cannabis useful for treating sleep apnea.

U.S. Patent Application No. 20150057342 discloses an oral formulation of cannabis compounds that provide an immediate release, a sustained release and a combination of a sustained and immediate release through an oil based cannabinoid formulation. These oral formulations required an additional banding step to prevent oil leakage from the capsules with an increased production cost.

There remains an unmet need for a measurable, reproducible oral dosage form of cannabinoid for the treatment of multiple clinical conditions where the cannabinoid is formulated in a lowcost capsule that provides extended shelf life and prevents oil leakage.

### SUMMARY OF THE INVENTION

The present invention discloses oral hard-fill formulations for the immediate-release of cannabinoids in order to avoid problems inherent with oil-based capsules containing cannabis.

An oral composition of cannabis obviates the need to smoke cannabis with the attendant hallucinatory effects associated with higher doses. The present invention has been developed for this purpose, and provides, in one aspect, a solid dosage form for oral administration, comprising: a cannabinoid or cannabinoids; a solvent into which the cannabinoid is solvated; and a carrier onto which the solvated cannabinoid is adsorbed.

In certain embodiments, the cannabinoid comprises delta-9-tetrahydrocannabinol (THC). In some embodiments, the THC is present at from about 0.1 mg to about 50 mg. In other embodiments, the cannabinoid is cannabidiol (CBD). In some embodiments, the CBD is present at from about 0.1 mg to about 200 mg.

One embodiment of the present invention incorporates the oral compositions of cannabinoids that provide an immediate-release, particularly the immediate-release of a combination of Tetrahydrocannabinol (THC) and Cannabidiol (CBD) in a powder form derived from a granulation utilizing microcrystalline cellulose as the carrier. HPMC capsules are used having a capsule size 2 with a 0.37 ml volume, an 18 mm locked length, and a 6.35 mm external diameter. The cannabinoids are easily prepared and formulated to provide consistent therapeutically effective dosage forms from lot to lot.

The preferred methods, uses, materials, and examples that will now be described are illustrative only and are not intended to be limiting; materials, uses and methods similar or equivalent to those described herein can be used in practice or testing of the invention. Other features and advantages of the invention will be apparent from the following detailed description, and from the claims.

### DESCRIPTION OF THE PREFERRED EMBODIMENTS

The present invention describes a therapeutic composition useful for orally administered cannabinoids. The composition incorporates, in part, a hard-fill formulation for the release of cannabinoids in order to avoid problems inherent with oil-based capsules containing cannabis. The present invention utilizes hard-fill formulations in providing a therapeutic window and eliminate the undesirable side effects associated with smoking or oil based capsules.

One embodiment of the present invention incorporates the oral compositions of cannabinoids to provide a release, particularly the sustained release of a combination of Tetrahydrocannabinol (THC) and Cannabidiol (CBD) in a powder form derived from a direct compression method utilizing microcrystalline cellulose as an excipient. HPMC capsules are used having a capsule size 2 with a 0.37 ml volume, an 18 mm locked length, and a 6.35 mm external diameter. The cannabinoids are easily prepared and formulated to provide consistent therapeutically effective dosage forms from lot to lot.

The inventors of the present invention unexpectedly discovered that a hard-fill oral composition which includes, in part, an extracted cannabinoid combined with microcrystalline cellulose in an HPMC capsule provides for the elimination of capsule leaking as seen in oil based capsules. Further the formulation disclosed herein reduces hallucinatory effects associated with large changes in dose.

One embodiment of the present invention is the HPMC size 2 capsules in an oral composition of cannabinoids comprising at least two cannabinoids in a powder form in a powder form derived from a direct compression method that utilizes microcrystalline cellulose as an excipient.

In some embodiments, the cannabinoid is a cannabinoid extract that contains a combination of at least two of the following: Tetrahydrocannabinol (THC), Cannabidiol (CBD), Cannabigerol (CBG), Cannabichromene (CBC), Cannabinol (CBN), Cannabielsoin (CBE), iso-Tetrahydrocannabimol (iso-THC), Cannabicyclol (CBL), Cannabicitran (CBT), Cannabivarin (CBV), Tetrahydrocannabivarin (THCV), Cannabidivarin (CBDV), Cannabichromevarin (CBCV), Cannabigerovarin (CBGV) and Cannabigerol Monomethyl Ether (CBGM) and derivatives thereof. The cannabinoid may be natural or synthetic.

In some embodiments, the cannabinoids are in equal proportion such as 2.5 mg THC to 2.5 mg CBD. Other embodiments include proportions of THC/CBD of 0.25 THC to 5.0 mg CBD or 5.0 mg THC to 0.25 mg CBD per capsule.

The compositions disclosed herein include a composition for daily administration. Preferably, the therapeutic effect of the capsules are maintained with one capsule, twice daily. The duration of each capsule's effect is between four (4) to six (6) hours.

In some embodiments, the cannabinoid reduces a symptom of a disease, treats a disease or attenuates side effects associated with the treatment. Disorders include, but not limited to, pain associated with cancer, neuropathic pain and HIV-associated sensory neuropathy, side effects of chemotherapy including nausea and pain, symptoms of neurology and neurodegenerative diseases such as Huntington's disease, Parkinson's disease, Alzheimer's disease, amyotrophic lateral sclerosis, multiple sclerosis, epilepsy, post-traumatic stress disorder (PTSD), alcohol abuse, bipolar disorder, depression, anorexia nervosa; cancer such as gliomas, leukemia, skin tumors, colorectal cancer; diseases including hepatitis C, methicillin-resistant Staphylococcus aureus (MRSA), pruritus, psoriasis, asthma, sickle-cell disease, sleep apnea, digestive diseases, collagen-induced arthritis, atherosclerosis and dystonia.

In some embodiments where the disorder is cancer, pain associated with cancer; nausea associated with chemotherapy; or a combination thereof, the composition described herein exerts reduced hallucinatory effects compared to smoking a cannabis containing cigarette or ingesting a cannabis containing foodstuff with the same amount of active ingredients.

In some embodiments, the solvent comprises ethanol, methanol, isopropanol, chloroform, propylene glycol, polyethylene glycol, glycerine, limonene, myrcene, linalool, alpha bisabolol, delta 3 carene, borneol, alpha-pinene, beta-pinene, eucalyptol, terpineol, caryophyllene, camphene, or combinations thereof.

In some embodiments, the carrier comprises cellulose, microcrystalline cellulose, silicified microcrystalline cellulose, starch, pregelatinized starch, dicalcium phosphate, tricalcium phosphate, and mixtures thereof. In other embodiments, the carrier may be comprised of a water-soluble sugar or sugar alcohol. Examples include, lactose, sucrose, dextrose, polydextrose, fructose, maltose, maltodextrin, dextrate, dextrin, lactitol, mannitol, erythritol, maltitol, sorbitol, or xylitol, and mixtures thereof.

In some embodiments, Cannabinoids of the present invention are any member of a group of substances that are structurally related to tetrahydrocannabinol and that bind to a cannabinoid receptor such as CB1 or CB2 or both (‘THC’). The cannabinoid can be a naturally occurring compound (e.g. present in Cannabis), a compound metabolized by a plant or animal, or a synthetic derivative.

The cannabinoid may be included in its free form, or in the form of a salt; an acid addition salt of an ester; an amide; an enantiomer; an isomer; a tautomer; a prodrug; a derivative of an active agent of the present invention; different isomeric forms (for example, enantiomers and diastereoisomers), both in pure form and in admixture, including racemic mixtures; enol forms.

The cannabinoids of the present invention are further meant to encompass natural cannabinoids, natural cannabinoids that have been purified or modified, and synthetically derived cannabinoids, for example, United States Patent Application Publication 2005/0266108, hereby incorporated by reference in its entirety, describes a method of purifying cannabinoids obtained from plant material.

The cannabinoids of the present invention can be any of 9-tetrahydrocannabinol, 8-tetrahydrocannabinol, (+)-1,1-dimethylheptyl analog of 7-hydroxy-delta-6-tetrahydrocannabinol, 3-(5′-cyano-1′,1′-dimethylpentyl)-1-(4-N-morpholinobutyryloxy) delta 8-tetrahydrocannabinol hydrochloride], dexanabinol, nabilone, levonantradol, or N-(2-hydroxyethyl) hexadecanoamide. The cannabinoids of the present invention can be any of the non-psychotropic cannabinoid 3-dimethylnepty 11 carboxylic acid homologine 8, delta-8-tetrahydrocannabinol.

In yet another aspect, provided herein is a method for the preparation of a sustained release, hard-fill formulation of capsules containing a combination of cannabinoids. The cannabis plants are grown, harvested, and the cannabinoids are extracted through a CO₂ extraction process. The cannabinoid extract is then combined with microcrystalline cellulose and ethanol in a high shear granulation process. The granules are then allowed to dry to remove the solvent. The dried granules are blended with sodium starch glycolate to form a powder blend which is encapsulated into a hard shell capsule (HPMC).

Exemplary formulations described above are shown in Table 1 and are prepared using the methods described herein.

TABLE 1

| | 20:1 | | 1:1 | | 1:20 | |
|---|---|---|---|---|---|---|
| | THC:CBD | | THC:CBD | | THC:CBD | |
| Material | % | mg/cap | % | mg/cap | % | mg/cap |
| THC | 3.33 | 5.0 | 1.67 | 2.5 | 0.17 | 0.25 |
| Cannabinoid | | | | | | |
| Extract | | | | | | |
| CBD | 0.17 | 0.25 | 1.67 | 2.5 | 3.33 | 5.0 |
| Cannabinoid | | | | | | |
| Extract | | | | | | |
| Micro- | 94.5 | 141.75 | 94.66 | 142.0 | 94.5 | 141.75 |
| crystalline | | | | | | |
| Cellulose | | | | | | |
| Sodium Starch | 2.0 | 3.0 | 2.0 | 3.0 | 2.0 | 3.0 |
| Glycolate | | | | | | |
| Ethanol¹ | | | | | | |
| Totals | 100 | 150 | 100 | 150 | 100 | 150 |

This manufacturing process of each individual capsule has an unexpectedly small variation from capsule to capsule, making this capsule and method of manufacture ideal in a large production environment. Manufacture of individual HPMC size 2 capsules have a maximum variation between each capsules in their target dose of approximately five percent (5%).

Unless defined otherwise, all technical and scientific terms used herein have the same meanings as commonly understood by one of ordinary skill in the art. Unique expressions used in the text are appropriately defined.

Those skilled in the art will recognize, or be able to ascertain using no more than routine experimentation, many equivalents to the specific embodiments of the invention described herein. Such equivalents are intended to be encompassed by the following claims.

1. An oral solid dosage form of cannabinoids, comprising:
a. dried granules comprising at least one cannabinoid extract from a cannabis plant and a pharmaceutically acceptable carrier;
b. sodium starch glycolate; and
c. a hard shell capsule,
wherein the dried granules are blended with the sodium starch glycolate to form a powder blend and the powder blend is encapsulated in the hard shell capsule.

2. The composition of claim 1 wherein the cannabinoid extract comprises at least two cannabinoids, wherein the two cannabinoids are selected from Tetrahydrocannabinol (THC), Cannabidiol (CBD), Cannabigerol (CBG), Cannabichromene (CBC), Cannabinol (CBN), Cannabielsoin (CBE), iso-Tetrahydrocannabimol (iso-THC), Cannabicyclol (CBL), Cannabicitran (CBT), Cannabivarin (CBV), Tetrahydrocannabivarin (THCV), Cannabidivarin (CBDV), Cannabichromevarin (CBCV), Cannabigerovarin (CBGV), Cannabigerol Monomethyl Ether (CBGM) and derivatives thereof.

3. (canceled)

4. The composition of claim 2 wherein the at least two cannabinoids are present in a 1:1 proportion by weight, a 10:1 proportion by weight, or a 20:1 proportion by weight.

5. (canceled)

6. (canceled)

7. The composition of claim 2 3- wherein the two cannabinoids are THC and CBD.

8. The composition of claim 1 wherein the one cannabinoid extract comprises CBD in an amount of approximately 0.1 mg to 200 mg.

9. (canceled)

10. The composition of claim 1 wherein the carrier comprises cellulose, microcrystalline cellulose, silicified microcrystalline cellulose, starch, pregelatinized starch, dicalcium phosphate, tricalcium phosphate, and mixtures thereof

11. The composition of claim 1 wherein the carrier comprises microcrystalline cellulose or a water-soluble sugar or sugar alcohol.

12. (canceled)

13. The composition of claim 1 wherein the hard shell capsule is hydroxypropyl methylcellulose (HPMC) or starch hydrolysate.

14. (canceled)

15. A method for treating a disease, comprising administering an oral solid dosage form of claim 1.

16. The method of claim 15, wherein the method comprises orally administering a single dosage form twice daily.

17. The method of claim 15, wherein the disease is selected from pain associated with cancer, neuropathic pain and HIV-associated sensory neuropathy, side effects of chemotherapy including nausea and pain, symptoms of neurology and neurodegenerative diseases such as Huntington's disease, Parkinson's disease, Alzheimer's disease, amyotrophic lateral sclerosis, multiple sclerosis, epilepsy, post-traumatic stress disorder (PTSD), alcohol abuse, bipolar disorder, depression, anorexia nervosa; cancer such as gliomas, leukemia, skin tumors, colorectal cancer; diseases including hepatitis C, methicillin-resistant Staphylococcus aureus (MRSA), pruritus, psoriasis, asthma, sickle-cell disease, sleep apnea, digestive diseases, collagen-induced arthritis, atherosclerosis and dystonia.

18. A method for the manufacture of hard shell capsules comprising the steps of:
a. combining at least one cannabinoid extract from a cannabis plant with a pharmaceutically acceptable carrier in a solvent to from granules using a high shear granulation means;
b. drying the granules;
c. blending the dried granules with sodium starch glycolate to form a powder blend; and
d. encapsulating the powder blend in a hard shell capsule.

19. (canceled)

20. The method of claim 18 wherein the cannabinoid extract comprises at least two cannabinoids selected from Tetrahydrocannabinol (THC), Cannabidiol (CBD), Cannabigerol (CBG), Cannabichromene (CBC), Cannabinol (CBN), Cannabielsoin (CBE), iso-Tetrahydrocannabimol (iso-THC), Cannabicyclol (CBL), Cannabicitran (CBT), Cannabivarin (CBV), Tetrahydrocannabivarin (THCV), Cannabidivarin (CBDV), Cannabichromevarin (CBCV), Cannabigerovarin (CBGV), Cannabigerol Monomethyl Ether (CBGM) and derivatives thereof.

21. The method of claim 20 wherein the two cannabinoids are present in a ratio selected from a group consisting of 1:1, 10:1, and 20:1.

22. The method of claim 20, wherein the two cannabinoids are THC and CBD.

23. The method of claim 18, wherein the cannabinoid extract comprises CBD in an amount of approximately 0.1 mg to 200 mg.

24. The method of claim 18,. wherein the pharmaceutically acceptable carrier is selected from cellulose, microcrystalline cellulose, silicified microcrystalline cellulose, starch, pregelatinized starch, dicalcium phosphate, tricalcium phosphate, and mixtures thereof.

25. The method of claim 24, wherein the pharmaceutically acceptable carrier comprises microcrystalline cellulose.

26. The method of claim 18, wherein the solvent is ethanol, methanol, isopropanol, chloroform, propylene glycol, polyethylene glycol, glycerine, limonene, myrcene, linalool, alpha bisabolol, delta 3 carene, borneol, alpha-pinene, beta-pinene, eucalyptol, terpineol, caryophyllene, camphene, or any combination thereof.

27. (canceled)

28. The method of claim 18 wherein the hard shell capsule is hydroxypropyl methylcellulose (HPMC) or starch hydrolysate.
